(19) United States
(12) Patent Application Publication
(10) Pub. No.: US 2008/0156071 A1
(43) Pub. Date: Jul. 3, 2008

(54) GAS SENSOR CALIBRATION FROM FLUID

(76) Inventor: Peter Tobias, Minnetonka, MN (US)

Correspondence Address: HONEYWELL INTERNATIONAL INC., 101 COLUMBIA ROAD, P O BOX 2245, MORRISTOWN, NJ, 07962-2245, US

(21) Appl. No.: 11/618,414

(22) Filed: Dec. 29, 2006

(51) Int. Cl. G01N 33/00

(52) U.S. Cl. 73/23.2; 73/1.06; 73/23.31; 73/1.03; 436/9

(57) ABSTRACT

Embodiments of the present invention relate to a gas sensor system comprising a gas sensor, a fluid reservoir enclosing a fluid and in proximity to the gas sensor and a membrane positioned between the gas sensor and fluid reservoir, wherein the membrane allows a sufficient amount of a test gas generated from the fluid to contact the gas sensor for testing. Embodiments also relate to a method of testing a gas sensor, the method comprises contacting reagents within a fluid reservoir, generating a test gas and contacting a gas sensor with the test gas sufficient to test the gas sensor.

### FIELD OF THE INVENTION

Embodiments of the present invention relate to gas sensor calibration from fluid and a gas sensor calibration system. More specifically, embodiments relate to gas sensor calibration utilizing a gas generated from a liquid.

### BACKGROUND

The reliability of toxic gas detectors is of great importance in many applications, especially when these instruments are used for ensuring the safety of personnel. Reliability is typically obtained by periodic checking of the instrument response to a test gas, however calibration test gases are typically supplied in large, bulky and expensive gas cylinders.

Potentially hazardous atmospheres are found in many locations, due to the presence of toxic gases, combustible gas mixtures or the excess or deficiency of oxygen concentration. Many types of gas detection instruments have been developed to provide a warning that the atmosphere contains potentially hazardous components, or to initiate remedial action. Examples of these gas detection instruments include the detection of combustible gases in coal mines, hydrogen sulfide in oil fields and water treatment plants, carbon monoxide in places ranging from steel mills to bedrooms, and oxygen in confined spaces, such as sewers. Within each gas detection instrument there are one or more gas sensors, whose function is to provide an electrical signal, which varies in response to the gas concentration.

Most gas sensors provide a relative output signal, such that the output signal is not an absolute measure of gas concentration, but merely proportional to the gas concentration. In such cases, the gas sensor must be calibrated with a known test gas prior to use. Calibration can also be used as a function check to ensure the sensor is working. The output from many types of sensors can vary over time and sensors can fail to operate without warning. Frequently calibrating a gas sensor can be time consuming, expensive and cumbersome in many applications. Calibrating a gas sensor is also limited to the reproducibility of the amount of test gas in contact with the sensor. Generating a gas from inhomogeneous solid materials introduces error in the reproducibility of a calibration pulse or amount of test gas produced.

### SUMMARY

Embodiments of the present invention relate to a gas sensor system comprising a gas sensor, a fluid reservoir enclosing a fluid and in proximity to the gas sensor and a membrane positioned between the gas sensor and fluid reservoir, wherein the membrane allows a sufficient amount of a test gas generated from the fluid to contact the gas sensor for testing. Embodiments also relate to a method of testing a gas sensor, the method comprises contacting reagents within a fluid reservoir, generating a test gas and contacting a gas sensor with the test gas sufficient to test the gas sensor.

### BRIEF DESCRIPTION OF THE DRAWINGS

FIG. 1 illustrates a schematic diagram of a gas sensor system 100 for calibrating a gas sensor using a heater, according to some embodiments.

FIG. 2 illustrates a schematic diagram of a gas sensor system 200 for calibrating a gas sensor utilizing a dispensable fluid reservoir, according to some embodiments.

FIG. 3 illustrates a schematic diagram of a gas sensor system 300 for calibrating a gas sensor utilizing a separate compartment for gas releasing materials, according to some embodiments.

FIG. 4 illustrates a block flow diagram of a method 400 of calibrating a gas sensor using a fluid, according to some embodiments.

### DETAILED DESCRIPTION

References in the specification to “one embodiment,” “an embodiment,” “an example embodiment,” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Embodiments of the present invention relate to a gas sensor system utilizing calibration from fluid. For the self-calibration of a gas sensor, a test gas may be generated nearby or adjacent to the sensor. By generating the test gas from a fluid, such as a liquid, the pulse of test gas may be much more predictable and consistent due to the better homogeneity of a fluid as compared to a solid.

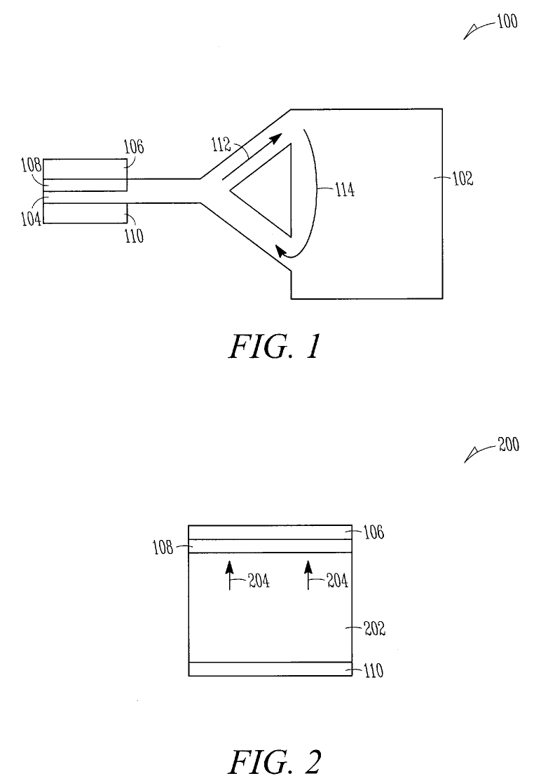

Referring to FIG. 1, a schematic diagram of a gas sensor system 100 for calibrating a gas sensor using a heater is shown, according to some embodiments. A gas sensor system 100 may include a larger fluid reservoir 102, enclosing a fluid. The fluid may cycle 114 to a smaller fluid reservoir 104 and be heated by a heater 110. Heating of the fluid may generate a test gas that passes through a membrane 108 to a gas sensor 106, for testing of the sensor. The fluid may cycle back 112 to the large reservoir.

The test gas may be released from the fluid by physical desorption or by chemical decomposition, for example. An example of physical desorption may include the generation of carbon dioxide and hydrogen sulfide from triethanolamine. An example of chemical decomposition may include carbon monoxide from glyoxal (H₂C₂O₂) in water. This reaction may also generate formaldehyde or hydrogen, which may stay in the fluid reservoir and not significantly affect the gas sensor.

The smaller fluid reservoir 104 may be utilized for gas generation as opposed to heating or generating gas from the larger fluid reservoir 102. By generating the test gas from the smaller fluid reservoir 104, less energy may be expended in heating or contacting reagents and conserves the starting reagents or gas releasing material, for example. The fluid in the small fluid reservoir 104 may be heated for a short period of time to generate a test gas, either depleting the small fluid reservoir 104 or allowing fluid to cycle back to the larger fluid reservoir 102. The fluid may cycle 112, 114 by convection, for example. The fluid may also cycle 112, 114 by active means, such as by utilizing a pump, for example.

A membrane 108 may be utilized to allow the passage of a test gas to a gas sensor, without fluid leaking out of the reservoir. The membrane may be very thin PTFE (polytetrafluoroethylene), for example. GORETEX® material may be an example of a suitable material used for the membrane 108.

The heater 110 may be a wire or thin film, for example. The heater 110 may be manufactured of or covered with a PTFE, such as TEFLON®, for example. The heater 110 may also be manufactured of or covered with a polyimide film, such as KAPTON®. The heater 110 may be made of a tungsten wire, for example. The heater 110 may be made of material inert to the test gas generating reaction or one that may be an intentional catalyst for the reaction, for example.

The gas sensor 106 may include a combustible gas sensor, for example. The gas sensor 106 may be a pellistor, for example. As the fluid is depleted, an optional piston may be utilized to maintain pressure with the reservoirs, for example.

Referring to FIG. 2, a schematic diagram of a gas sensor system 200 for calibrating a gas sensor utilizing a dispensable fluid reservoir is shown, according to some embodiments. A gas sensor system 200 may include a fluid reservoir 202, enclosing a fluid. A heater 110 may heat the fluid within the fluid reservoir 202, generating a test gas that passes 204 through a membrane 108 to a gas sensor 106, for testing. The fluid within the fluid reservoir 202 may be entirely depleted to generate a test gas, for example. An optional piston may be utilized to replace the diminishing volume and maintain pressure.

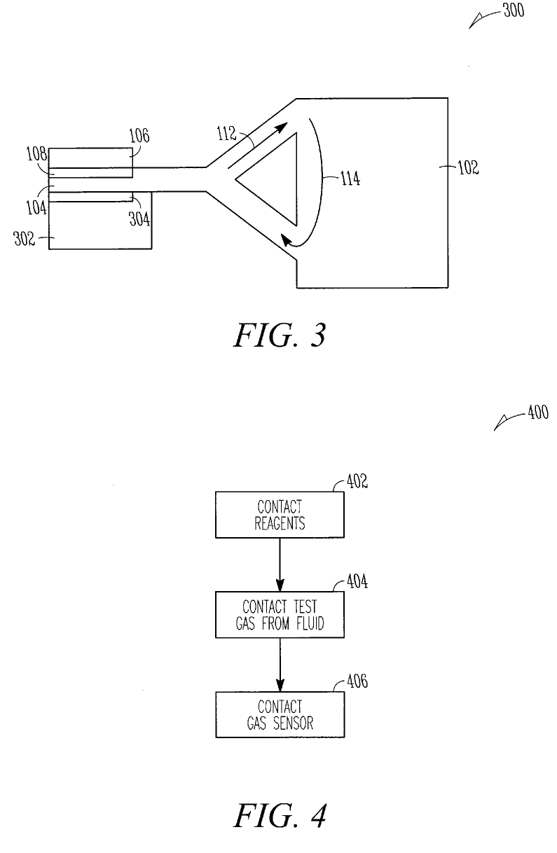

Referring to FIG. 3, a schematic diagram of a gas sensor system 300 for calibrating a gas sensor utilizing a separate compartment for gas releasing materials is shown, according to some embodiments. A gas sensor system 300 may include a larger fluid reservoir 102, enclosing a fluid. The fluid may cycle 114 to a smaller fluid reservoir 104. A separate compartment 302 may enclose a reagent that when in contact with the fluid, generates a test gas. A mechanism, such as a valve 304, may control the release of the reagent from the compartment 302. The generated test gas then passes through a membrane 108 to a gas sensor 106, for testing of the sensor. The fluid may cycle back 112 to the large reservoir.

The mechanism to separate the smaller fluid reservoir 104, such as a valve 304, from the large fluid reservoir 102 may also be a separator that collapses or that may be destroyed by heating. Methane would be an example of test gas that may be generated from a liquid without the utilization of a heater. Methane may be generated by the reaction

Al₄C₃+6H₂O=>2Al₂O₃+3CH₄.

The aluminum carbide may be held in the separate compartment 302 and released to the smaller fluid reservoir 104 to initiate the gas releasing reaction, for example.

Referring to FIG. 4, a block flow diagram of a method 400 of calibrating a gas sensor using a fluid is shown, according to some embodiments. Reagents may be contacted 402 within a fluid reservoir. A test gas may then be generated 404 from the reagents, one or more of which may be a fluid. The generated test gas may then contact a gas sensor 406, sufficient to test the gas sensor.

The test gas may be generated 404 by mixing a gas releasing material or reagents or by heating a gas releasing material, for example. Contacting 402, 406 may include mixing or exposing, for example.

The test gas may contact the gas sensor, sufficient to test the target sensor. The test may be a bump test or a calibration test. The bump test exposes a high enough concentration of the test gas to the sensor for the sensor alarm to trigger, effectively testing the functionality of the sensor. A calibration provides a concentration suitable to reset the baseline concentration, effectively correcting for any drift or contamination in the sensor. The calibration or bump test may be activated as often as desired, with the only limitation being the amount of gas releasing material available or any electrical or battery power limitations involved with activating the heater. The tests may be performed every few minutes, hourly, daily, weekly, etc.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1. A gas sensor system comprising:
a gas sensor;
a fluid reservoir, enclosing a fluid and in proximity to the gas sensor; and
a membrane, positioned between the gas sensor and fluid reservoir;
wherein the membrane allows a sufficient amount of a test gas generated from the fluid to contact the gas sensor for testing.

2. The gas sensor system of claim 1, wherein the gas sensor comprises a combustible gas sensor.

3. The gas sensor system of claim 1, wherein the gas sensor comprises a pellistor.

4. The gas sensor system of claim 1, wherein fluid comprises a liquid.

5. The gas sensor system of claim 1, further comprising a heater, positioned near or within the fluid reservoir.

6. The gas sensor system of claim 5, wherein the heater comprises a wire.

7. The gas sensor system of claim 6, wherein the wire comprises tungsten.

8. The gas sensor system of claim 5, wherein the wire is covered by PTFE.

9. The gas sensor system of claim 1, wherein the fluid comprises one or more gas releasing materials.

10. The gas sensor system of claim 1, wherein the fluid reservoir comprises a smaller fluid reservoir and a larger fluid reservoir, in contact with each other.

11. The gas sensor system of claim 10, wherein the smaller fluid reservoir comprises an amount of the fluid sufficient to generate an amount of test gas.

12. The gas sensor system of claim 1, wherein the test gas comprises methane.

13. The gas sensor system of claim 1, wherein the test gas comprises carbon monoxide.

14. A gas sensor system comprising:
a pellistor;
a fluid reservoir, enclosing a fluid and in proximity to the gas sensor; and
a PTFE membrane, positioned between the gas sensor and fluid reservoir;
wherein the membrane allows a sufficient amount of a test gas generated from the fluid to contact the pellistor for calibration testing.

15. A method of testing a gas sensor, the method comprising:
contacting reagents within a fluid reservoir;
generating a test gas from a fluid; and
contacting a gas sensor with the test gas, sufficient to test the gas sensor.

16. The method of claim 15, wherein contacting reagents comprises mixing.

17. The method of claim 15, wherein generating a test gas comprises heating the reagents.
